(19) United States
(12) Patent Application Publication
(10) Pub. No.: US 2018/0146905 A1
(43) Pub. Date: May 31, 2018

(54) PERSONALITY TESTING DEVICE AND METHOD

(71) Applicant: TWINWORD INC., Seongnam-si (KR)

(72) Inventors: Kun Oh KIM, Anyang-si (KR); Woo Young CHUN, Daejeon (KR); Namhee KIM, Daejeon (KR); Goeun SHIN, Daejeon (KR); Hye Jin RYU, Daejeon (KR)

(73) Assignee: TWINWORD INC., Seongnam-si (KR)

(21) Appl. No.: 15/575,196

(22) PCT Filed: May 10, 2016
(86) PCT No.: PCT/KR2016/004891

§ 371 Date: Nov. 17, 2017

(30) Foreign Application Priority Data
May 19, 2015 (KR) 10-2015-0069740

(51) Int. Cl. A61B 5/16; G09B 7/02; G09B 7/077; G09B 19/00

(52) U.S. Cl. CPC A61B 5/167; G09B 19/00; G09B 7/077; G09B 7/02

(57) ABSTRACT

Disclosed are personality testing device and method. A personality testing device, according to an embodiment of the present invention, comprises: an input and output unit for providing test questions which comprise a stimulation word and obtaining a response word which is a response received from a person to be tested with respect to the stimulation word; and a control unit for forming the test questions by extracting a stimulation word from a word graph that is classified by personality types and for distinguishing the personality type of the person to be tested by analyzing the response word by means of the word graph.

### CROSS REFERENCE TO RELATED APPLICATIONS

This application claims the benefit under 35 USC 119(a) of PCT Application No. PCT/KR2016/004891, filed on May 10, 2016, which claims the benefit of Korean Patent Application No. 10-2015-0069740 filed on May 19, 2015, in the Korean Intellectual Property Office, the entire disclosure of which is incorporated herein by reference for all purposes.

### TECHNICAL FIELD

The present invention relates to information service technology, and more particularly, to technology for testing a user's psychology and providing checked information.

### BACKGROUND ART

Psychological tests are measurement methods, whereby human intelligence, aptitude, personality or the like is tested and a test result is represented by systematic values. The psychological tests include various types of psychological tests according to test purposes, such as an intelligence test for measuring a cognitive ability, an aptitude test for measuring an aptitude ability, an achievement test for measuring an ability for studies, and a personality test for measuring personality such as personal characteristics, attitude or temperament. A person can be known more precisely by such a psychological test, and checked information can be used in various ways according to their usage purposes. Through the personality test among the psychological tests, a typical personality type is systematically determined based on a predetermined principle, and various personalities can be classified and arranged by the personality test such that understanding of personality can be facilitated.

### DISCLOSURE

### Technical Problem

The present invention is directed to providing an apparatus for testing personality, in which a testee's personality can be easily, simply and interestingly determined, and a method thereof.

### Technical Solution

One aspect of the present invention provides an apparatus for testing personality including: an input/output unit configured to provide a test question including a stimulus word and to acquire a response word returned from a testee with respect to the stimulus word; and a controller configuring a test question by extracting the stimulus word from a word graph classified according to a personality type, configured to analyze the response word using the word graph, and to determine the testee's personality type.

The word graph may be classified data by grouping words having a meaning distance within a predetermined range according to keywords that represent personality types, and the meaning distance may be a physical distance indicated based on similarity of meaning between the words.

The controller according to an embodiment may configure a test question that suggests a stimulus word and requests the testee of freely inputting a first word that comes into his/her mind with respect to the suggested stimulus word, and may determine the testee's personality type according to a response word returned from the testee with respect to the test question.

The controller according to an embodiment may configure a test question that suggests stimulus words and requests the testee of selecting a word that is more representative to him/her, from among the suggested stimulus words, and may determine the testee's personality type according to a response word selected from the testee with respect to the test question.

The controller according to an embodiment may configure a test question that suggests stimulus words and requests the testee of displaying a slide with a word that is more representative to him/her, from among the suggested stimulus words, and may determine the testee's personality type according to a response word slide-selected from the testee with respect to the test question.

The controller according to an embodiment may configure a test question that suggests words relating to a stimulus word together with the stimulus word and requests the testee of selecting a word that comes into his/her mind with respect to the stimulus word, from among the suggested words, and may determine the testee's personality type according to a response word selected from the testee with respect to the test question.

The controller according to an embodiment may configure a test question that suggests words relating to a stimulus word together with the stimulus word and requests the testee of displaying a slide with a word that comes into his/her mind with respect to the stimulus word, from among the suggested words, and may determine the testee's personality type according to a response word slide-selected from the testee with respect to the test question.

The controller according to an embodiment may calculate a meaning distance between a stimulus word and a response word by referring to a word graph and may determine the testee's personality according to the calculated meaning distance. Alternatively, the controller may determine the testee's personality type by projecting response words with respect to a stimulus word into a word graph classified according to personality types and analyzing a distribution of the response words projected into the word graph.

The controller according to an embodiment may extract stimulus words randomly from the word graph and may extract at least one stimulus word for each of classified keywords. The controller according to an embodiment may configure a test question by using an unconsciousness psychological testing method, whereby the testee responds to a stimulus word with a response word associated by his/her unconsciousness. The controller according to an embodiment may reflect the testee's response word with respect to a stimulus word so as to extract a next stimulus word from the word graph. The controller may provide a plurality of test types having different test questions and test times so that the testee is capable of selecting a test type.

### ADVANTAGEOUS EFFECTS

According to an embodiment, a testee can know his/her own personality easily, simply and interestingly without an expert's help. That is, a psychological test according to the present invention is a simple testing method and has a small number of test questions so that the testee can know his/her own personality within a short time. In particular, a test can be interestingly carried out by a testing method, such as a free association method, a word selection method, and a slider method. Furthermore, the testee's complex can be known, and the testee's personality can be determined.

In a personality test according to the present invention, a meaning distance between a stimulus word and a response word is analyzed using a word graph classified according to personality types, or the distribution of response words projected into the word graph is analyzed so that the testee's personality type can be determined and a comparatively precise result can be provided. Also, the testee's unconsciousness can be more scientifically interpreted using an unconsciousness psychological testing method. Furthermore, various additional services can be provided using the result of determination of the testee's personality type.

### DESCRIPTION OF DRAWINGS

FIG. 1 is a view of a configuration of a personality testing system according to an embodiment of the present invention.

FIG. 2 is a view of a configuration of a personality testing system according to another embodiment of the present invention.

FIG. 3 is a view of a configuration of an apparatus for testing personality according to an embodiment of the present invention.

FIG. 4 is a detailed view of a configuration of a controller of FIG. 3, according to an embodiment of the present invention.

FIGS. 5 through 9 are reference views of personality testing screens according to various embodiments of the present invention.

FIG. 10 is a reference view of a personality testing result screen according to an embodiment of the present invention.

FIG. 11 is a flowchart illustrating a method of testing personality according to an embodiment of the present invention.

FIG. 12 is a reference view of a part of a word graph according to an embodiment of the present invention.

### MODES OF THE INVENTION

The present invention will now be described more fully with reference to the accompanying drawings, in which exemplary embodiments of the invention are shown. In the description of the present invention, if it is determined that a detailed description of commonly-used technologies or structures related to the invention may unnecessarily obscure the subject matter of the invention, the detailed description will be omitted. Also, since later-described terms are defined in consideration of the functions of the present invention, they may vary according to a user, an operator's intention or practice. Hence, the terms must be interpreted based on the contents of the entire specification.

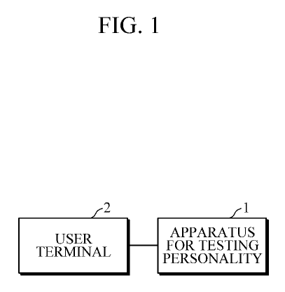

FIG. 1 is a view of a configuration of a personality testing system according to an embodiment of the present invention.

An apparatus 1 for testing personality according to an embodiment suggests a stimulus word to a testee, analyzes a response word to be returned from the testee with respect to the stimulus word, thereby determining the testee's personality type. In this case, the response word is a word associated by the testee with respect to the stimulus word. When the apparatus 1 for testing personality suggests a test question including the stimulus word, the testee answers the stimulus word with the response word, and the apparatus 1 for testing personality determines the testee's personality type using the response word. A test provided by the apparatus 1 for testing personality is a method, which requires no intervention of an expert, such as a psychologist and the testee's personality type can be easily and simply known without investing much time or cost.

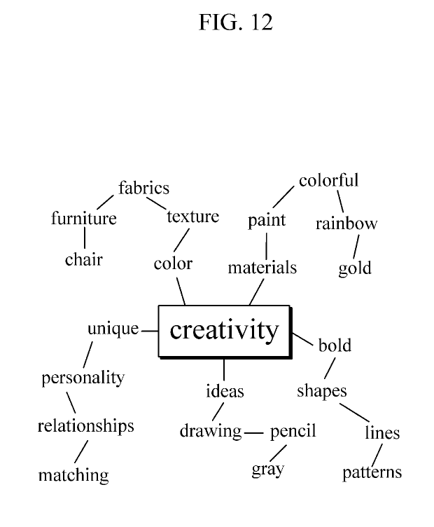

The apparatus 1 for testing personality according to an embodiment determines the testee's personality type using a word graph. For example, the testee's personality type can be determined by analyzing a meaning distance between the stimulus word and the response word, or by analyzing the distribution of response words projected into the word graph, for example. The word graph is classified data by grouping words having a meaning distance in a predetermined range according to keywords. Keywords for the personality type may include eight words, such as extraversion, introversion, sensing, intuition, thinking, feeling, judging, and perceiving. In this case, the word graph is composed of words having the meaning distance in the predetermined range according to the eight keywords. The meaning distance is indicated by a physical distance based on similarity of meaning between words. As illustrated in FIG. 12, the words are arranged close to or far from each other to configure the word graph, and the words may have similarity of meaning with several words.

The psychological test provided by the apparatus 1 for testing personality uses a word association testing method using a response word associated with respect to a stimulus word. One of word association testing methods used in analytic psychology is made by C. G. Jung who selects 100 stimulus words having the maximum frequency based on a F. W. Kaeding's frequency dictionary established in 1908 and lists nouns, adjectives or adverbs alternately and randomly. Although the word association testing method is not usually used, an interest therein is recently increasing in consideration of the research into the correlation between mind and body. This test is not a psychological test for a statistical research or diagnosis but is used as a tool for arousing and conscientizing the testee's unconscious complex in the testee's analysis procedure.

Hereinafter, features of a personality test according to the present invention distinguished from a psychological test based on the C. G. Jung's word association will be described.

The C. G. Jung's word association-based psychological test is a test for measuring the testee's complex based on a response speed at which the response word is drawn with respect to the stimulus word. In order to measure this, an expert is required. In this case, in order to measure the testee's complex, there is an inconvenience that a tester has to do a test with the same stimulus word several times. The C. G. Jung's psychological test is not a test for determining the testee's personality but a test for measuring the testee's complex.

The testee by himself/herself can easily and simply carry out the word association-based personality test according to the present invention without the expert's help, unlike in the C. G. Jung's psychological test, and in the word association-based personality test according to the present invention, the testee's unconsciousness can be scientifically interpreted, and the testee's personality type can be checked.

In detail, the testee by himself/herself can know his/her own personality without a trained expert's help by using the personality test according to the present invention. For example, a testing method is easy and simple, like in a method of suggesting two stimulus words and selecting a word that is more representative to him/her, from among the two stimulus words, or of expressing a degree of preference of the word using a slide. Also, because the number of test questions is not large so that the testee can know his/her own personality through a test within a short time. There is a difference between the C. G. Jung's word association testing method and the personality test according to the present invention in that the C. G. Jung's word association testing method is used to check the testee's complex, whereas, in the personality test according to the present invention, not only the testee's complex can be checked, but also the testee's personality can be determined.

Meanwhile, in the personality test according to the present invention, the testee's unconsciousness can be more scientifically interpreted, no expert's help is required, and a testing method is simple and interesting, compared to a Myers-Briggs type indicator (MBTI) that is one personality testing method.

In detail, test questions in the MBTI are pre-set but, in the personality test according to the present invention, test questions are randomly determined. Also, in case of the MBTI, the number of test questions is 90 or more so that it takes long to carry out a test. However, the number of questions in the personality test according to the present invention is very small. For example, the apparatus 1 for testing personality constitutes questions by randomly extracting stimulus words from the word graph and extracting at least one stimulus word for each of 8 keywords classified. In this case, the number of test questions is minimum eight. When it is assumed that three questions per keyword are suggested in consideration of test accuracy, the total number of questions is 24 (8×3) and thus is much smaller than the number of 90 or more questions of the MBTI. Furthermore, the apparatus 1 for testing personality may select a stimulus word to be suggested next time by seeing the testee's response word so as to increase test accuracy. In this case, a predetermined number of questions, for example, about 8 questions are added to 24 questions so that the total number of questions is 32, and the personality can be checked using only 32 questions, and the number of 32 questions is still much smaller than the number of 90 questions of the MBTI. The smaller the number of test questions is, the shorter the testee's testing time is.

The personality test provided by the apparatus 1 for testing personality according to an embodiment may provide a plurality of test types having different test questions and test times so that the testee can select a test type. For example, simply, a test type including 32 questions is provided as a basic type, and when the testee wants to know his/her own personality more precisely, the testee only needs to select a test type including more questions. When the testee wants to know his/her own personality within a shorter time, the testee may also select a test type including a smaller number of questions.

The personality test according to the present invention is a test for checking the testee's inner world using an unconsciousness psychological testing method. The unconsciousness psychological testing method is a method, whereby the testee inputs a response word associated by his/her unconsciousness with respect to a stimulus word. For example, in the personality test according to the present invention, a testee is required to respond to a singer with a first word that comes into the testee's mind. In this case, the testee responds without knowing a tester's intention properly. When the testee knows the tester's intention, the testee can respond to a test in a distorted manner so that the reliability of the test is lowered. Thus, the unconsciousness psychological testing method may increase the reliability of the test.

The apparatus 1 for testing personality according to an embodiment provides various additional services using the result of judging of the testee's personality type. For example, the testee's personality is analyzed based on the result of judging of the testee's personality type, and a psychological treatment service can be provided. The testee can check ego through psychological status analysis and can use short-term and long-term psychological treatment services according to the psychological status. In another example, recommendation information or advertisement information customized for the testee can be provided according to the testee's personality type. For example, a particular product or service can be recommended according to the testee's personality. To this end, the apparatus 1 for testing personality may be connected to an advertisement server, etc.

Hereinafter, the configuration of a personality testing system according to an embodiment will be described with reference to FIG. 1. Referring to FIG. 1, the personality testing system includes the apparatus 1 for testing personality and a user terminal 2.

The testee has access to the apparatus 1 for testing personality via a wired/wireless network using the user terminal 2 that the testee holds, so as to carry out a test provided by the apparatus 1 for testing personality on the user terminal 2. A test program may be executed in the user terminal 2 for a test. The user terminal 2 has access to the apparatus 1 for testing personality via the Internet so as to carry out the test provided by the apparatus 1 for testing personality and to receive the result of the test. To this end, the apparatus 1 for testing personality may directly provide a web page to which the user terminal 2 is connectable, or may be connected to an additional web server so as to provide a web page to the user terminal 2. When the apparatus 1 for testing personality determines the testee's personality type, the user terminal 2 receives the result of determination from the apparatus 1 for testing personality and displays the received result on a display screen.

The user terminal 2 that is a device capable of executing the test program may include various types of devices, such as a personal computer (PC), a mobile phone, a smartphone, a personal digital assistant (PDA), a tablet computer, a netbook, an IPTV, a portable multimedia player (PMP), a navigation device, an MP3 player, a mobile broadcasting receiver, a digital camera, and the like. The apparatus 1 for testing personality manages a word graph required to determine the testee's personality type. To this end, the apparatus 1 for testing personality may use the word graph stored in an additional server and may include a word graph database having the word graph stored in the apparatus 1 for resting personality.

The configuration of the system described with reference to FIG. 1 is illustrative, and the system may further include other devices required for an operation. For example, information exchange between the user terminal 2 and the apparatus 1 for testing personality may be performed via an additional wired or wireless communication network, and a communication device for communication between the user terminal 2 and the apparatus 1 for testing personality may be additionally provided.

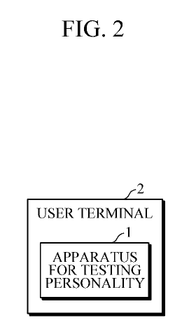

FIG. 2 is a view of a configuration of a personality testing system according to another embodiment of the present invention.

Referring to FIG. 2, the personality testing system may be a system in which the user terminal 2 and the apparatus 1 for testing personality are integrally formed. For example, as illustrated in FIG. 2, the apparatus 1 for testing personality may be included in the user terminal 2. In this case, the apparatus 1 for testing personality may use the function of the user terminal 2. In another example, the user terminal 2 itself may be replaced with the apparatus 1 for testing personality.

The apparatus 1 for testing personality analyzes the response word returned from the testee, determines the testee's personality type, and displays the result of determination of the personality type on the display screen of the user terminal 2. The apparatus 1 for testing personality may use the word graph required to determine the personality type from the database stored in an additional server. When a word graph database is stored in the apparatus 1 for testing personality, the word graph database may be used.

The apparatus 1 for testing personality may be an additional apparatus separated from the user terminal 2, as illustrated in FIG. 1, and may be an apparatus integrated with the user terminal 2, as illustrated in FIG. 2. However, some elements of the apparatus 1 for testing personality may be included in the user terminal 2 if necessary, and the remaining elements thereof may be placed in the additional apparatus separated from the user terminal 2.

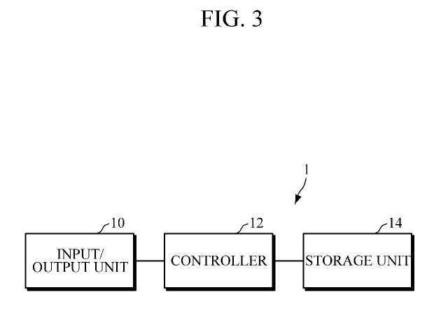

FIG. 3 is a view of a configuration of an apparatus for testing personality according to an embodiment of the present invention.

The configuration of the apparatus 1 for testing personality shown in FIG. 3 is illustrative, and the apparatus 1 for testing personality may include only a part of the elements illustrated in FIG. 3 and/or may further include other elements required for an operation of the apparatus 1 for testing personality. Hereinafter, each of the elements of the apparatus 1 for testing personality will be described in detail with reference to FIG. 3.

Referring to FIG. 3, the apparatus 1 for testing personality includes an input/output unit 10 and a controller 12 and may further include a storage unit 14.

The input/output unit 10 receives data or outputs the data to transmit the output data to another device so that the apparatus 1 for testing personality can be interlocked with other devices. For example, when the user terminal requests the apparatus 1 for testing personality of provision of information about the testee's personality, the input/output unit 10 receives requested data and transmits the received data to the controller 12, and when the testee's personality type is determined by the controller 12, the input/output unit 10 receives the result of determination from the controller 12 and transmits the received result of determination to the user terminal.

The controller 12 that is a kind of processor controls an overall operation of the apparatus 1 for testing personality. The controller 12 extracts a stimulus word from the word graph classified according to the personality type so as to configure test questions and analyzes responses using the word graph so as to determine the testee's personality type.

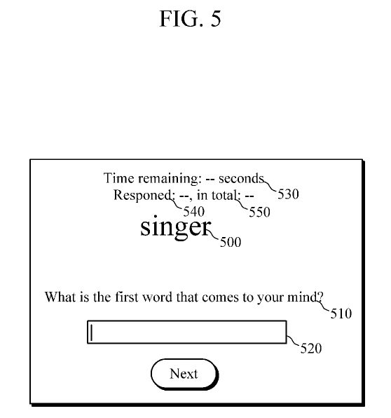

The controller 12 provides various testing methods. For example, the controller 12 may provide free association, word selection, a slider method, or the like. Various embodiments thereof will be descried later with reference to FIGS. 5 through 9.

The controller 12 according to an embodiment extracts stimulus words randomly from the word graph and suggests the stimulus words. In this case, the controller 12 may extract at least one stimulus word for each of the classified keywords. For example, the stimulus words are extracted and suggested according to 8 keywords, such as extraversion, introversion, sensing, intuition, thinking, feeling, judging, and perceiving.

The controller 12 according to an embodiment analyzes a meaning distance between the stimulus word and the response word using the word graph, thereby determining the testee's personality type. In an example, a meaning distance between the stimulus word and the response word is calculated by referring to the word graph, and a percentage of correct answers or a percentage of wrong answers is extracted according to the calculated meaning distance, and the testee's personality is determined using the extracted percentage of correct answers or the extracted percentage of wrong answers. Correct answers or wrong answers may be determined according to the meaning distance. The case where the testee responds to the stimulus word with the response word within a predetermined meaning distance, is determined as a correct answer, and the case where the testee responds with the response word out of the predetermined meaning distance, is determined as a wrong answer. In the wrong answer, another stimulus word may be extracted from the word graph of the personal type in which the stimulus word that causes the wrong answer is extracted, and may be suggested to the testee so that the testee's response can be additionally checked.

In another example, the controller 12 projects response words with respect to the stimulus word into the word graph classified according to the personality type, analyzes a distribution of the response words in the word graph, and determines the testee's personality type. In an example, stimulus words are suggested from a word graph classified based on different keywords, for example, a word graph classified based on 8 keywords, such as extraversion, introversion, sensing, intuition, thinking, feeling, judging, and perceiving, and response words selected from the testee with respect to the suggested stimulus words are analyzed so that the testee's personality type can be determined. For example, a stimulus word extracted from a word graph relating to extroversion and a stimulus word extracted from a word graph relating to introversion are suggested together. In this case, when the testee selects the stimulus word extracted from the word graph relating to introversion, it is determined that the testee is an introverted person. In another example, the controller 12 analyzes the distribution of the response words in the word graph classified based on 8 keywords, and determines the testee's personality type according to the response words concentrated on which word graph.

Various information required by the apparatus 1 for testing personality so as to provide a personality test service is stored in the storage unit 16. The storage unit 16 may transfer the stored information to the controller 12. The word graph may be stored in the storage unit 16. The storage unit 16 may be a storage medium including a memory, a hard disk drive (HDD), or the like but is not limited thereto.

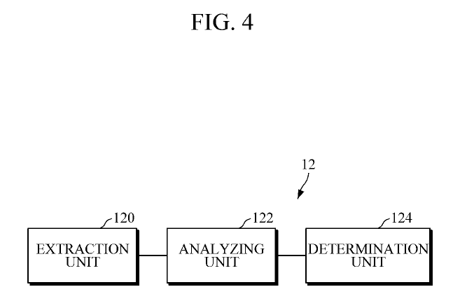

FIG. 4 is a detailed view of a configuration of the controller of FIG. 3, according to an embodiment of the present invention.

Referring to FIGS. 3 and 4, the controller 12 includes an extraction unit 120, an analyzing unit 122, and a determination unit 124.

The extraction unit 120 extracts stimulus words from the word graph classified according to the personality type, thereby configuring test questions. The extraction unit 120 may extract the stimulus words from the word graph randomly and may extract at least one stimulus word for each of classified keywords. The extraction unit 120 may configure the test questions using the unconsciousness psychological testing method, whereby the testee responds to the stimulus word with the response word associated by his/her unconsciousness. The extraction unit 120 may extract a next stimulus word from the word graph by reflecting the testee's response word with respect to the stimulus word.

The analyzing unit 122 analyzes the response word using the word graph, and the determination unit 124 determines the testee's personality type using the result of analysis of the analyzing unit 122.

In the explanation of the personality type in order to assist in understanding of determination of the personality type of the determination unit 124, the personality type may be an extroversion-introversion (E-I) index indicating directivity of mental energy, a sense-intuition (S-N) index indicating the function of perceiving including information gathering, a thinking-feeling (T-F) index for reasonably judging and determining based on the gathered information, a judgment-perceiving (J-P) index indicating a life style in which a perceiving function and a judging function are revealed in a real life. The testee's personality can be checked through a form having combined 8 preference indices.

In detail, the E-I index is an index indicating whether directivity of psychological energy and concern is introverted or extroverted. The S-N index is an index indicating which one between sensing and intuition is more usually used, in a method of perceiving and recognizing an object such as a person or an object. The T-F index indicates which one between thinking and feeling is more preferred when judging and determination is made based on the gathered information. The J-P index is an index indicating a tendency for preference of one between judging and perceiving in a method of coping with a real life based on the perceiving function and the judging function.

According to the result based on the above-described 8 classification criteria, the testee's personality may be classified into one among 16 personality types, such as ISTJ, ISFJ, INFJ, INTJ, ISTP, ISFP, INFP, INTP, ESTP, ESFP, ENFP, ENTP, ESTJ, ESFJ, ENFJ, and ENTJ, which are used in the MBTI or the Jung personality type. A most preferred function among the perceiving function (S or N) and the determination function (T or F) in these 16 personality types is referred to as a main function, and a function for assisting the main function is referred to as an auxiliary function. For example, when the main function is a T type, the auxiliary function is naturally one perceiving function (S or N). That is, the 16 personality types have different main functions and auxiliary functions, and they are divided according to the directivity of mental energy introverted or extroverted and have different characteristics.

FIGS. 5 through 9 are reference views of a personality testing screen according to various embodiments of the present invention.

In detail, FIG. 5 illustrates a personality testing screen in a free association testing method according to an embodiment. Referring to FIG. 5, the apparatus for testing personality provides a test question that suggests a stimulus word 500 and requests (510) the testee of freely inputting the first word that comes to his/her mind with respect to the stimulus word 500. With respect to this, the testee inputs the first response word 520 that comes to his/her mind. In this case, the apparatus for testing personality may give an input limit time per question, for example, 40 seconds, and may display the remaining time 530 on a screen. Also, the apparatus for testing personality may display information (responded; 540) about an order of this question and information about the total number of questions (in total; 550) on the screen.

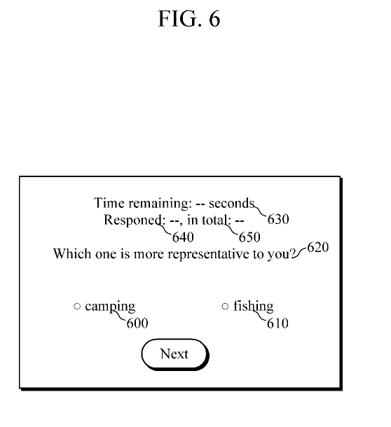

FIG. 6 illustrates a personality testing screen in a word selection testing method according to an embodiment. Referring to FIG. 6, the apparatus for testing personality provides a test question that suggests stimulus words 600 and 610 and requests (620) the testee of selecting a word that is more representative to him/her, from among the suggested stimulus words 600 and 610. With respect to this, the testee selects one from among the stimulus words 600 and 610. In this case, the apparatus for testing personality may give an input limit time per question, for example, 40 seconds, and may display the remaining time (630) on the screen. Also, the apparatus for testing personality may display information about an order of this question (responded; 640) and information about a total number of questions (in total; 650) on the screen.

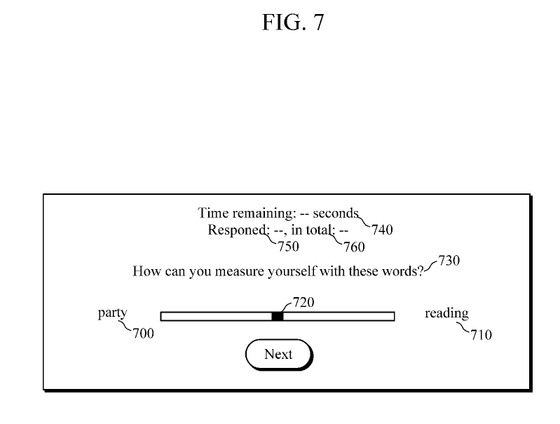

FIG. 7 illustrates a personality testing screen in a slider testing method according to an embodiment. Referring to FIG. 7, the apparatus for testing personality provides a test question that suggests stimulus words 700 and 710 and requests (730) the testee of displaying a slide 720 with a word that is more representative to him/her, from among the suggested stimulus words 700 and 710. With respect to this, the testee displays a slide with a word that is more representative to him/her, from among the stimulus words 700 and 710. The slider method is a method, whereby a degree of preference can be together known compared to the above selection method described with reference to FIG. 6. In this case, the apparatus for testing personality may give an input limit time per question, for example, 40 seconds, and may display the remaining time (740) on the screen. Also, the apparatus for testing personality may display information about an order of this question (responded; 750) and information about a total number of questions (in total; 760) on the screen.

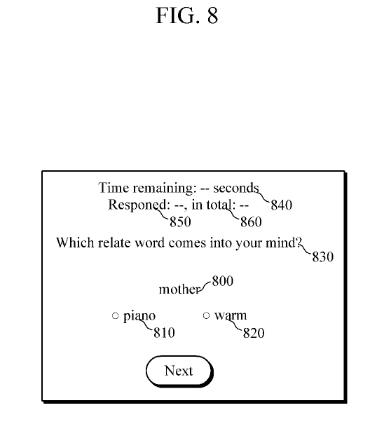

FIG. 8 illustrates a personality testing screen in a word selection testing method according to another embodiment. Referring to FIG. 8, the apparatus for testing personality provides a test question that suggests words 810 and 820 relating to a stimulus word 800 together with the stimulus word 800 and requests (830) the testee of selecting a word that comes into his/her mind with respect to the stimulus word 800, from among the suggested words 810 and 820. With respect to this, the testee selects one from among the suggested words 810 and 820. In this case, the apparatus for testing personality may give an input limit time per question, for example, 40 seconds, and may display the remaining time (840) on the screen. Also, the apparatus for testing personality may display information about an order of this question (responded; 850) and information about a total number of questions (in total; 860).

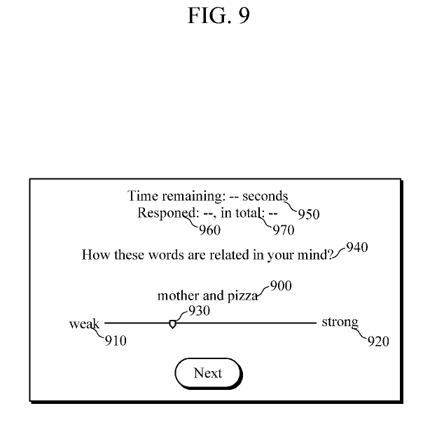

FIG. 9 illustrates a personality testing screen in a slider testing method according to another embodiment. Referring to FIG. 9, the apparatus for testing personality provides a test question that suggests words 910 and 920 relating to a stimulus word 900 together with the stimulus word 900 and requests (940) the testee of displaying a slide 930 with a word that comes into his/her mind with respect to the stimulus word 900, from among the suggested words 910 and 920. With respect to this, the testee displays a slide with a word that is more representative to him/her, from among the suggested words 910 and 920. In this case, the apparatus for testing personality may give an input limit time per question, for example, 40 seconds, and may display the remaining time 950 on the screen. Also, the apparatus for testing personality may display information about an order of this question (responded; 960) and information about a total number of questions (in total; 970).

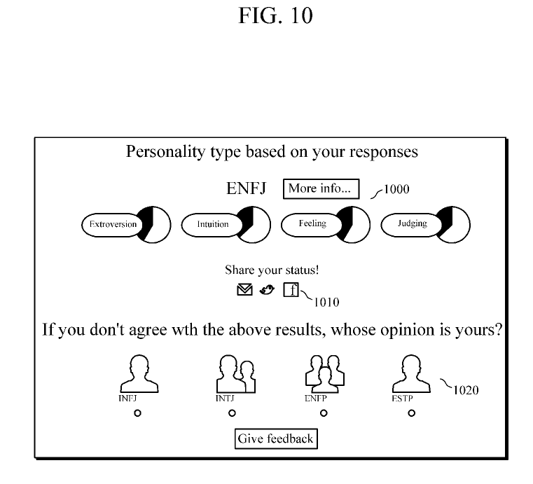

FIG. 10 is a reference view of a personality testing result screen according to an embodiment.

Referring to FIG. 10, the apparatus for testing personality displays a personality testing result 1000. As illustrated in FIG. 10, the apparatus for testing personality classifies the testee's personality into one of 16 personality types used in the MBTI, such as, ISTJ, ISFJ, INFJ, INTJ, ISTP, ISFP, INFP, INTP, ESTP, ESFP, ENFP, ENTP, ESTJ, ESFJ, ENFJ, and ENTJ, using 8 indices such as extraversion, introversion, sensing, intuition, thinking, feeling, and judging. However, this is just an embodiment of the present invention, and a method of classifying personality is not limited thereto.

The apparatus for testing personality provides a screen 1010 on which the testee's personality testing result can be shared. For example, the testee may upload the personality testing result on an e-mail, a twitter, a facebook, or the like. Also, the apparatus for testing personality may receive feedback about the personality testing result from the testee. For example, when the testee does not agree with the personality testing result, the apparatus for testing personality may provide a screen 1020 through which the testee's opinion can be heard.

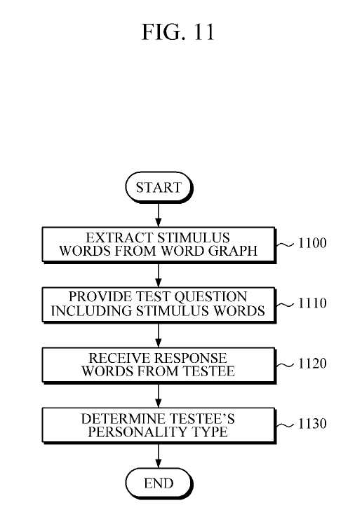

FIG. 11 is a flowchart illustrating a method of testing personality according to an embodiment of the present invention.

Referring to FIG. 11, the apparatus for testing personality extracts stimulus words from a word graph classified according to a personality type (Operation 1100), and configures and provides a test question including the extracted stimulus words (Operation 1110). Subsequently, the apparatus for testing personality receives response words returned from the testee with respect to the stimulus words (Operation 1120), and analyzes the response words using the received word graph and determines the testee's personality type (Operation 1130).

The word graph is data classified by grouping words having a meaning distance within a predetermined range according to keywords that represent personality types, and the meaning distance is a physical distance indicated based on similarity of meaning between the words.

In Operation 1100 of extracting the stimulus words, the apparatus for testing personality may extract stimulus words randomly from the word graph and may extract at least one stimulus word for each of the classified keywords. Also, in Operation 1100 of extracting the stimulus words, the testee's response words with respect to the stimulus words are reflected, and the next stimulus word may be extracted from the word graph.

In Operation 1110 of configuring and providing a test question including the stimulus words, the apparatus for testing personality may configure a test question that suggests a stimulus word and requests the testee of inputting the first word that comes into his/her mind with respect to the suggested stimulus word. Alternatively, the apparatus for testing personality may configure a test question that suggests stimulus words and requests the testee of selecting a word that is more representative to him/her, from among the suggested stimulus words. Alternatively, the apparatus for testing personality may configure a test question that suggests stimulus words and requests the testee of displaying a slide with a word that is more representative to him/her, from among the suggested stimulus words. Alternatively, the apparatus for testing personality may configure a test question that suggests words relating to a stimulus word together with the stimulus word and requests the testee of selecting a word that comes into his/her mind with respect to the stimulus word, from among the suggested words. Alternatively, the apparatus for testing personality may configure a test question that suggests words relating to a stimulus word together with the stimulus word and requests the testee of displaying a slide with a word that comes into his/her mind with respect to the stimulus word, from among the suggested words.

In Operation 1110 of configuring and providing the test question including the stimulus words, the apparatus for testing personality may configure a test question using an unconsciousness psychological testing method, whereby the testee responds to the stimulus words with response words associated by his/her unconsciousness.

In Operation 1130 of determining the testee's personality type, the apparatus for testing personality may calculate a meaning distance between a stimulus word and a response word by referring to the word graph and may determine the testee's personality according to the calculated meaning distance. In this case, the apparatus for testing personality may extract a percentage of correct answers or a percentage of wrong answers according to the calculated meaning distance and may determine the testee's personality using the extracted percentage of correct answers or the extracted percentage of wrong answers. In another example, the apparatus for testing personality may determine the testee's personality type by projecting response words with respect to the stimulus word into the word graph classified according to the personality type and analyzing a distribution of the response words projected into the word graph.

FIG. 12 is a reference view of a part of the word graph according to an embodiment of the present invention.

Referring to FIG. 12, the word graph is a meaning network between words, and words are arranged relatively far from or close to each other in the word graph according to a meaning distance between the words. A testing apparatus is used to check the testee's psychological state reflected in a written text by distinguishing a meaning relationship and a meaning distance between the words of the word graph. Although a tiny part of the word graph is shown in FIG. 12, the word graph may have a map or graph shape with an enormous size including a lot of words.

While the invention has been shown and described with reference to certain exemplary embodiments thereof, it will be understood by those skilled in the art that various changes in form and details may be made therein without departing from the essential characteristics of the invention. Therefore, the exemplary embodiments should be considered in a descriptive sense only and not for purposes of limitation. Furthermore, the technical scope of present invention is defined not by the detailed description of the invention but by the appended claims, and all differences within the scope will be construed as being comprised in the present invention.

1. An apparatus for testing personality, comprising:
an input/output unit configured to provide a test question including a stimulus word and to acquire a response word returned from a testee with respect to the stimulus word; and
a controller configuring a test question by extracting the stimulus word from a word graph classified according to a personality type, configured to analyze the response word using the word graph, and to determine the testee's personality type.

2. The apparatus of claim 1, wherein the word graph is classified data by grouping words having a meaning distance within a predetermined range according to keywords that represent personality types, and
the meaning distance is a physical distance indicated based on similarity of meaning between the words.

3. The apparatus of claim 1, wherein the controller configures a test question that suggests a stimulus word and requests the testee of freely inputting a first word that comes into his/her mind with respect to the suggested stimulus word, and determines the testee's personality type according to a response word returned from the testee with respect to the test question.

4. The apparatus of claim 1, wherein the controller configures a test question that suggests stimulus words and requests the testee of selecting a word that is more representative to him/her, from among the suggested stimulus words, and determines the testee's personality type according to a response word selected from the testee with respect to the test question.

5. The apparatus of claim 1, wherein the controller configures a test question that suggests words relating to a stimulus word together with the stimulus word and requests the testee of selecting a word that comes into his/her mind with respect to the stimulus word, from among the suggested words, and determines the testee's personality type according to a response word selected from the testee with respect to the test question.

6. The apparatus of claim 1, wherein the controller configures a test question that suggests stimulus words and requests the testee of displaying a slide with a word that is more representative to him/her, from among the suggested stimulus words, and determines the testee's personality type according to a response word slide-selected from the testee with respect to the test question.

7. The apparatus of claim 1, wherein the controller configures a test question that suggests words relating to a stimulus word together with the stimulus word and requests the testee of displaying a slide with a word that comes into his/her mind with respect to the stimulus word, from among the suggested words, and determines the testee's personality type according to a response word slide-selected from the testee with respect to the test question.

8. The apparatus of claim 1, wherein the controller calculates a meaning distance between a stimulus word and a response word by referring to a word graph and determines the testee's personality according to the calculated meaning distance.

9. The apparatus of claim 1, wherein the controller determines the testee's personality type by projecting response words with respect to a stimulus word into a word graph classified according to personality types and analyzing a distribution of the response words projected into the word graph.

10. The apparatus of claim 1, wherein the controller extracts stimulus words randomly from the word graph and extracts at least one stimulus word for each of classified keywords.

11. The apparatus of claim 1, wherein the controller configures a test question by using an unconsciousness psychological testing method, whereby the testee responds to a stimulus word with a response word associated by his/her unconsciousness.

12. The apparatus of claim 1, wherein the controller reflects the testee's response word with respect to a stimulus word so as to extract a next stimulus word from the word graph.

13. The apparatus of claim 1, wherein the controller provides a plurality of test types having different test questions and test times so that the testee is capable of selecting a test type.
